(19) United States
(12) Patent Application Publication
(10) Pub. No.: US 2018/0273898 A1
(43) Pub. Date: Sep. 27, 2018

(54) BIOLOGICAL MATERIAL AND METHOD OF MANUFACTURING THE SAME

(71) Applicant: MAY-HWA ENTERPRISE CORPORATION, TAIPEI CITY (TW)

(72) Inventors: Hsien-Yeh Chen, Taipei City (TW); Zhen-Yu Guan, Taipei City (TW)

(21) Appl. No.: 15/587,386

(22) Filed: May 4, 2017

(30) Foreign Application Priority Data
Mar. 22, 2017 (TW) 106109610

(51) Int. Cl. C12N 5/00; A61L 27/54; A61L 27/34; C12N 5/0775

(52) U.S. Cl. CPC C12N 5/0018; A61L 27/54; A61L 27/34; C12N 5/0667; C12N 2533/30; C12N 2533/50; A61L 2420/00; C12N 2533/90; C12N 2500/30; C12N 2500/34; A61L 2300/412; A61L 2300/414; A61L 2400/18; C12N 2533/52

(57) ABSTRACT

The present invention provides a biological material including a parylene C film; and first proteins which are adsorbed on the surface of the parylene C film. According to an embodiment of the present invention, the biological material further includes second proteins different from the first proteins adsorbed on the surface of the parylene C film. According to an embodiment of the present invention, the first proteins or the second proteins include BMP-2, fibronectin or PRP.

### BACKGROUND OF THE INVENTION

### 1. Field of the Invention

The present invention relates to a biological material. More particularly, the present invention relates to a modified parylene-C film and a method of manufacturing the same.

### 2. Description of the Prior Art

Polychloro-para-xylylene, commercially known as parylene-C, has gained FDA approval (United States Pharmacopeia, Class VI polymer) as a coating for biomedical applications. These types of coatings effectively protect the underlying surface or device from corrosion or oxidation during long-term exposure to a hostile environment of extracellular fluids. The advantages of parylene-C, such as its chemical and biological inertness, ability to block oxygen and vapors, slippery surface texture, and excellent electrical insulation, have been reviewed in other publications. The key features of being inert and protective when coated on other materials allow parylene-C to meet the stringent requirements of material interfaces for use in sophisticated biological environments. Biocompatibility is an indisputable requirement in advanced biomaterial surface design; in addition, biomaterials often need to serve a specific biological function when applied, and their design can be tailored toward functions such as mimicking the multifunctional interface of the extracellular matrix to promote stem cell differentiation and proliferation. It is challenging to modify parylene-C to exhibit such properties, however. Although some post-modifications, e.g., post chemical grafting, can be sporadically applied to parylene-C surfaces to induce biological effects, potentially harmful solvents and chemical substances are involved in the modification processes.

In the surface modification of implantable biological materials, many past studies have aimed to covalently bind a stable, specific biomolecule. Producing a stable chemical bond with a biomolecule means that the chemical reaction will take longer, which does not meet the needs of real medical surgery, where every second counts. Therefore, achieving rapid and effective surface modification has become the biggest advantage of the physical adsorption as compared to the chemical covalent bond.

Many studies have been performed to elucidate protein-surface interactions and provide guidance in designing new biomaterials. Consensus on the detailed mechanism of protein adsorption has not yet been achieved and engineering approaches for manipulating protein molecules on material interfaces have not been established. Although the adsorption of molecules on material surfaces is a basic and intuitively understood phenomenon, the mechanisms governing protein adsorption are complex. Current technologies may provide solutions to some of these problems and enable the creation of a biological environment that prevents further adsorption of non-specific proteins, widely known as non-fouling surfaces. Consideration of protein adsorption tends to be limited to prevention in the design of new biomaterials, and improving biocompatibility appears to be the only useful application of protein adsorption.

If, however, the physical adsorption is not effectively controlled, many biomolecules will not be selectively adsorbed, causing much interference by other biological effects. Recently, the main purpose for non-specific physical adsorption of biomolecules has been directed to anti-stick, anti-scaling and other technologies. Adsorption of biomolecules is not entirely a bad thing, if effective and accurate control of adsorption of specific biomolecules on the biological materials surface, can make the surface be modified with the biological function of the adsorbed molecules in a short time. The ability of controlling protein and interface properties will facilitate the processing of biomaterials for clinical application and industrial products.

### SUMMARY OF THE INVENTION

The present invention provides a novel method for manufacturing a biological material, wherein the method introduces a different functional material, such as bone morphogenic protein-2 (BMP-2), fibronectin and platelet-rich plasma (PRP), to the surface of parylene-C via the simple and intuitive process of protein adsorption.

The present invention in one aspect provides a biological material including a parylene-C film and first proteins, wherein the first proteins are adsorbed on a surface of the parylene-C film. According to an embodiment of the present invention, the biological material further comprises second proteins different from the first proteins adsorbed on the surface of the parylene-C film. According to an embodiment of the present invention, the first proteins or the second proteins include BMP-2, fibronectin or PRP.

The present invention in another aspect provides a method of manufacturing a biological material. The method includes the steps of: providing a substrate; performing a vapor deposition process such that a parylene-C film is deposited on the substrate; and providing a protein solution, wherein the parylene-C film is immersed in the protein solution to form a surface substance on the parylene-C film. According to an embodiment of the present invention, the method further includes a rinse process to rinse the surface substance after forming the surface substance on the parylene-C film. According to an embodiment of the present invention, the surface substance has biological functions. According to an embodiment of the present invention, the biological functions include cell proliferation and/or osteogenesis. According to an embodiment of the present invention, the protein solution comprises two or more proteins and has a predetermined ratio, wherein a composition ratio of the surface substance is the same as the predetermined ratio of the protein solution. According to an embodiment of the present invention, the rinse process includes the steps of: rinsing the surface substance three times with a phosphate-buffered saline containing Tween-20; rinsing the surface substance once with a phosphate-buffered saline without Tween-20; and rinsing the surface substance once with deionized water. According to an embodiment of the present invention, the surface substance is adsorbed on the parylene-C film and the adsorption of the surface substance is irreversible.

The present invention in another aspect provides a use of a biological material according to claim 1, wherein the biological material is used for cell culture.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 illustrates steps of manufacturing a biological material according to the present invention.

FIG. 2A and FIG. 2B are Infrared reflection-absorption spectroscopy (IRRAS) spectra of a biological material according to the first embodiment of the present invention.

FIG. 3 and FIG. 4 are quartz crystal microbalance (QCM) analyses of a biological material according to the first embodiment of the present invention.

FIG. 5 is a QCM analysis of a biological material according to the second embodiment of the present invention.

FIG. 6A and FIG. 6B are QCM analyses of the binding affinity of a specific antibody according to the third embodiment of the present invention.

FIG. 7A and FIG. 7B are optical microscopic images of cell culture after 24 hours and 72 hours, respectively, according to the fifth embodiment of the present invention.

FIG. 8A is an ALP expression after 10 days of cell culture according to the sixth embodiment of the present invention.

FIG. 8B is an optical microscope image of calcium mineralization after 21 days of cell culture according to the sixth embodiment of the present invention.

### DETAILED DESCRIPTION

In the following detailed description of the disclosure, reference is made to the accompanying drawings, which form a part hereof, and in which is shown, by way of illustration, specific embodiments in which the invention may be practiced. These embodiments are described in sufficient detail to enable those skilled in the art to practice the invention. Other embodiments may be utilized and structural changes may be made without departing from the scope of the present disclosure.

To provide a better understanding of the presented invention, preferred embodiments will be made in detail. The preferred embodiments of the present invention are illustrated in the accompanying drawings with numbered elements.

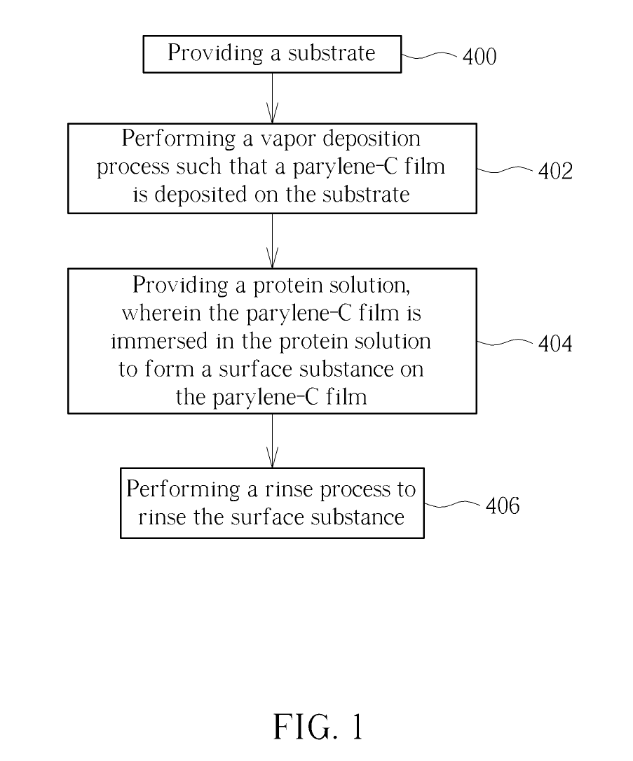

FIG. 1 illustrates steps of manufacturing a biological material according to the present invention. As shown in FIG. 1, the method of the present invention includes the following steps:

Step 400: providing a substrate;

Step 402: performing a vapor deposition process such that a parylene-C film is deposited on the substrate;

Step 404: providing a protein solution, wherein the parylene-C film is immersed in the protein solution to form a surface substance on the parylene-C film; and

Step 406: performing a rinse process to rinse the surface substance.

The following describes each step.

First, a substrate is provided (Step 400). In an embodiment, the substrate may be stainless steel, titanium, gold, and tissue culture polystyrene (TCPS). TCPS is used in cell culture experiments, and is well recognized as a good culture substrate.

Next, a vapor deposition process is performed such that a parylene-C film is deposited on the substrate (Step 402). The polychloro-para-xylylene (parylene-C) film is prepared by a custom-built chemical vapor deposition (CVD) system comprising a sublimation zone, a pyrolysis furnace, and a deposition chamber.

During the CVD polymerization process, dichloro-[2,2]-paracyclophane is first vaporized at approximately 150° C. and then transported to the pyrolysis furnace, where the dimer is pyrolyzed into monomer radicals at 670° C. The radicals then enter the deposition chamber and are polymerized on a rotating holder maintained at 15° C. to form a uniform parylene-C film. To inhibit residual deposition, the temperature of the chamber wall is maintained at 90° C. A stream of argon at a flow rate of 25 sccm is used as a carrier gas. Throughout the CVD polymerization, the operation pressure is regulated at 75 mTorr, and the deposition rate is maintained at approximately 0.5 Å/s. The parylene-C film formed by the present invention has the formula (1), wherein n refers to an integral greater than 750,000.

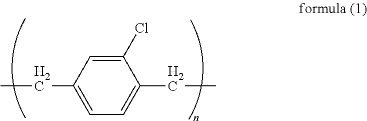

Next, a protein solution is provided, wherein the parylene-C film is immersed in the protein solution to form a surface substance on the parylene-C film (Step 404). The protein solution may be any solution containing functional proteins. In one embodiment, the protein solution includes bone morphogenic protein-2 (BMP-2), fibronectin and platelet-rich plasma (PRP), but is not limited thereto. The surface substance is formed by immersing coated parylene-C film substrate in the protein solution at 4° C. for 10 minutes to adsorb the functional protein in the protein solution onto the surface of the parylene-C film. The adsorption time of 10 minutes in the present invention is used to ensure the equilibrium has been reached rather than a critical requirement to perform such protein adsorption for BMP-2, fibronectin or PRP. In one embodiment, the protein solution may be a mixed solution including two functional proteins and has a predetermined ratio. The mixing ratio is adjusted based on the mass concentration, and a composition ratio of the surface substance is the same as the predetermined ratio of the protein solution.

A quartz crystal microbalance (QCM) analysis is performed with an ADS-QCM instrument equipped with a flow injection analysis (FIA) device and a continuous frequency variation recording device. The flow rate is controlled by using a peristaltic pump connected to the FIA device. The sensing element of this instrument is an AT-cut piezoelectric quartz disc with a 9 MHz resonant frequency and a 0.1 cm² total sensing area. The quartz crystal sensors are coated with parylene-C film via the previously described CVD polymerization process. After a stable frequency response is obtained, the selected protein solutions are injected through the FIA device to the analysis chamber, and the time-dependent change in frequency is continuously monitored. All the experiments are carried out at 25° C. The term “adsorption” as used in the present invention refers to the amount of protein adsorbed measured by quantitatively analyzing QCM, where the results show that the frequency range is between 18 Hz (Hz) and 350 Hz (Hz), indicating that the protein has been successfully adsorbed on a target surface.

Finally, a rinse process is performed to rinse the surface substance (Step 406). The rinse process includes the steps of: rinsing the surface substance three times with a phosphate-buffered saline containing Tween-20; rinsing the surface substance once with a phosphate-buffered saline without Tween-20; and rinsing the surface substance once with deionized water. The rinse process removes the loosely adsorbed proteins.

The above-mentioned biological material may be used for cell culture by adsorbing a surface substance including functional proteins on the surface of the parylene-C film to induce the biological function of the cell, for example, cell proliferation or osteogenesis. The cell culture may be used in vivo or in vitro. The model system of multifunctional surfaces featuring adsorbed fibronectin, BMP-2 and PRP displays synergistic and concurrent multifunctions of cell proliferation and osteogenesis independently induced by fibronectin or BMP-2 and PRP.

Hereinafter, the embodiments of the present invention will be described in detail.

### Example 1

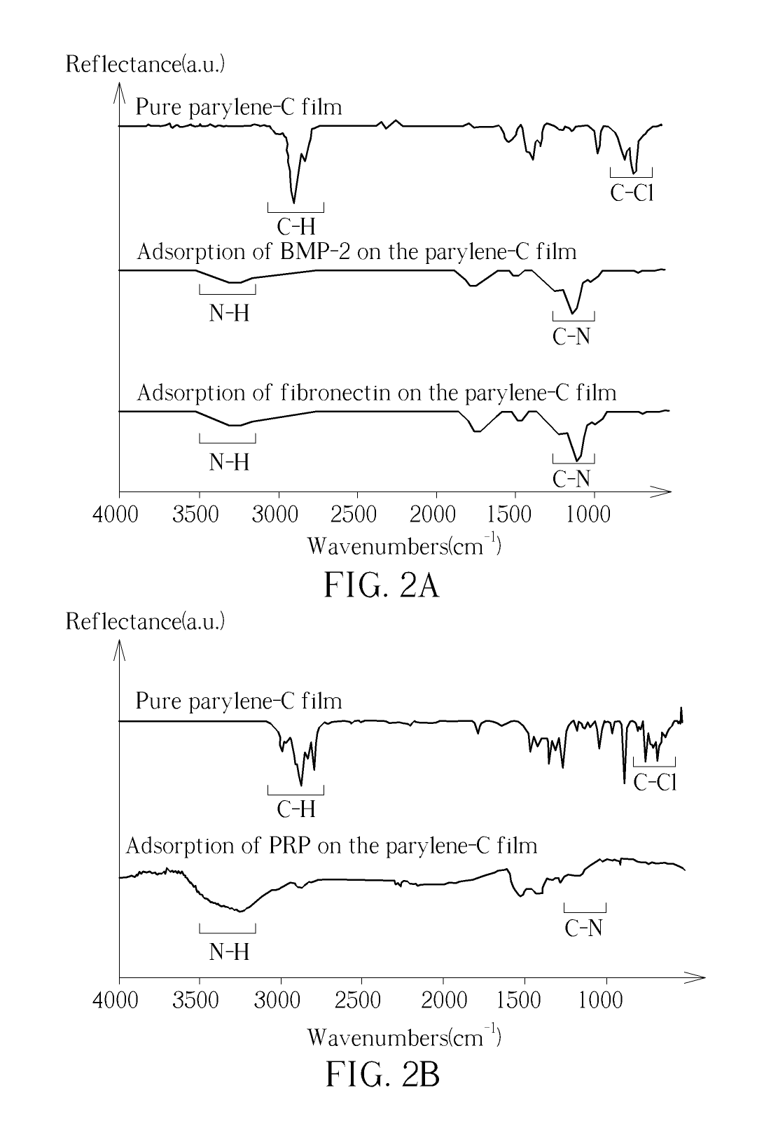

In the present embodiment, the surface substance of the parylene-C film is formed by immersing parylene-C film in a protein solution containing a single class of protein. Since the protein solution containing a single class of protein is used, the composition of the surface substance has only one functional protein. FIG. 2A and FIG. 2B are Infrared reflection-absorption spectroscopy (IRRAS) spectra of a biological material according to the first embodiment of the present invention. The samples are mounted in a nitrogen-purged chamber. Each spectrum is obtained from the acquisition of 128 scans at 4 cm⁻¹ resolution from 500 to 4000 cm⁻¹. As indicated in FIG. 2A and FIG. 2B, compared with the spectrum of pure parylene-C film, characteristic N—H bands at 3300-3500 cm⁻¹ and C—N bands at 1000-1250 cm⁻¹ are detected after the adsorption of BMP-2, fibronectin and PRP onto the surfaces of the parylene-C film. This demonstrates that the functional proteins are successfully adsorbed on the surface of the parylene-C film.

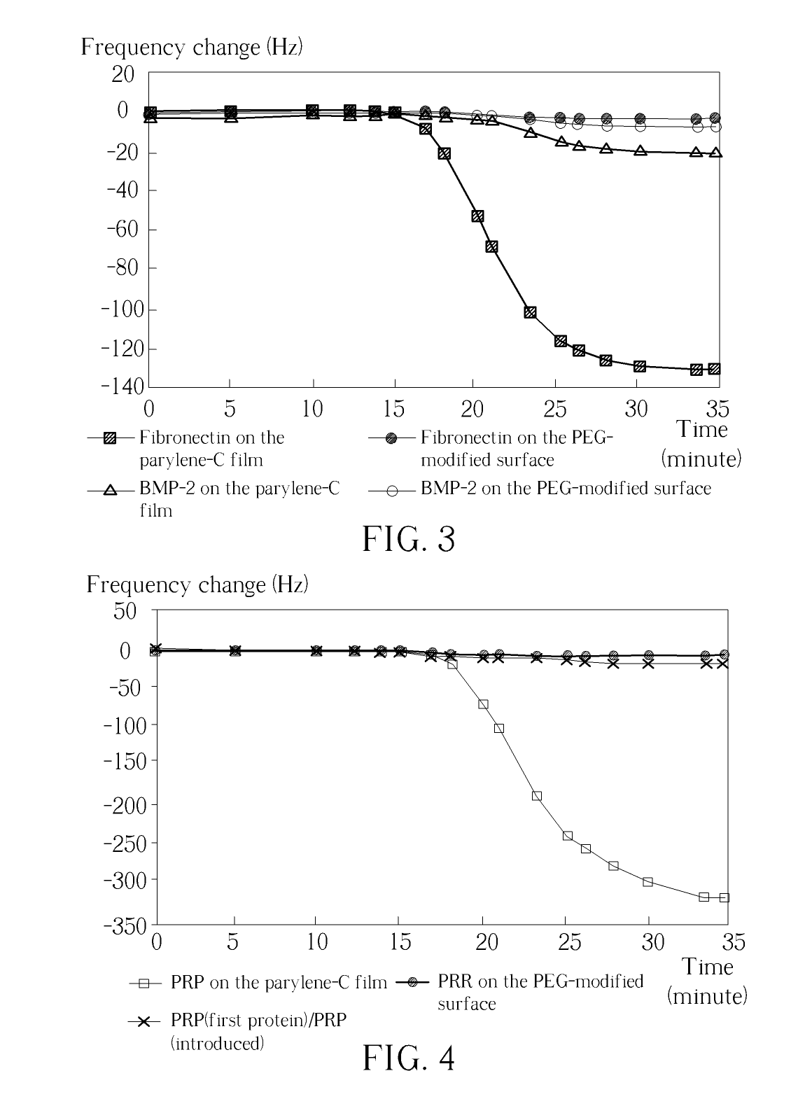

FIG. 3 and FIG. 4 are quartz crystal microbalance (QCM) analyses of a biological material according to the first embodiment of the present invention, wherein the horizontal axis represents the time (minute) and the vertical axis represents the frequency change (Hz). Polyethylene glycol (PEG)-modified surface is shown to be protein resistant, thereby preventing nonspecific protein adsorption, and is used as a control surface where low protein adsorption is expected. Concerning the QCM analyses of fibronectin and BMP-2 adsorbed on the surface of the parylene-C film, as shown in FIG. 3, the observed frequency changes are 130.4±11.9 Hz for fibronectin and 19.6±1.2 Hz for BMP-2. These results correspond to approximately 64.7±5.9 ng/cm² of fibronectin and 9.7±0.6 ng/cm² of BMP-2 that have been adsorbed onto the surfaces of the parylene-C film. The adsorption of low fibronectin (1.5+−0.5 ng/cm²) and BMP-2 (3.4+−1.1 ng/cm²) is observed with respect to PEG-modified surfaces. Concerning the QCM analyses of the PRP adsorbed on the surface of the parylene-C film, as shown in FIG. 4, the observed frequency changes is 340±11.9 Hz, which corresponds to 168.9±5.9 ng/cm² of PRP that have been adsorbed onto the surfaces of the parylene-C film. These results confirm that the successful adsorption of these proteins onto the surfaces of the parylene-C film.

### Example 2

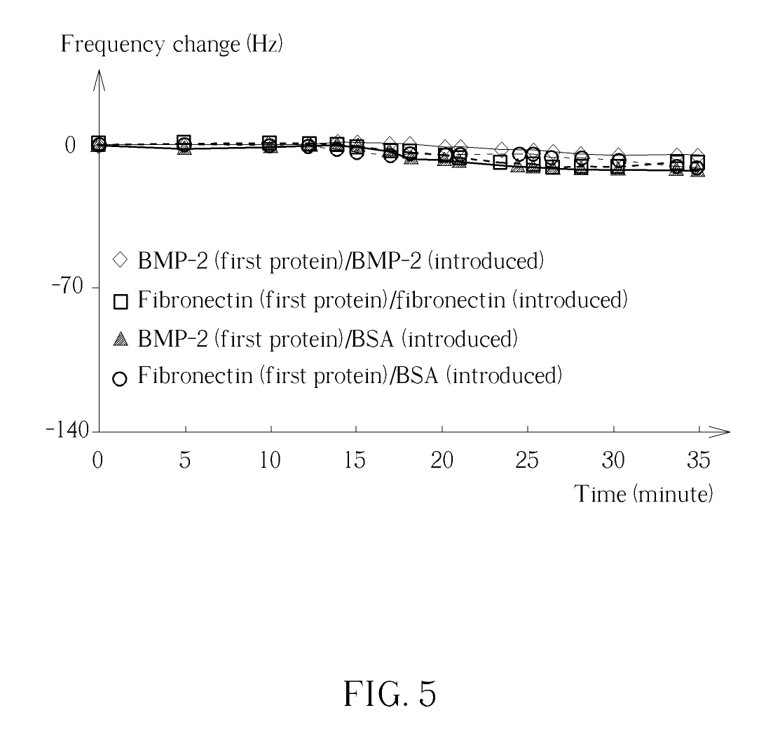

In the present embodiment, QCM analysis is further used to characterize the adsorption stability for surfaces on which BMP-2 or fibronectin layers have already been adsorbed (first protein surface). Homologous fibronectin (BMP-2 or fibronectin) or heterologous (BSA, 66 kDa) proteins are introduced to the first protein surface to dynamically investigate the binding affinity. Please refer to FIG. 5, illustrating QCM analysis is used to characterize the subsequent adsorption affinity QCM analysis for the surfaces on which BMP-2 or fibronectin layers were already adsorbed. As shown in FIG. 5, low adsorption affinities ranging from 2.9±0.3 ng/cm² to 5.9±0.3 ng/cm² are detected for BMP-2, fibronectin, and BSA to adsorb on the first protein surface. Concerning a QCM analysis of the subsequent adsorption affinity for the surface on which PRP layer is already adsorbed as shown in FIG. 4, low frequency change 15.8±2.9 Hz is detected for PRP. These results indicate that (i) the previously adsorbed BMP-2 or fibronectin saturate the adsorption capacity, preventing further adsorption of protein molecules; (ii) low fouling property of the stable interface is established for the first adsorbed BMP-2 or fibronectin surface, and the resistance to subsequent protein adsorption is non-specific (irrespective of homologous and heterologous protein types); and (iii) the previous adsorption of BMP-2 or fibronectin is irreversible.

### Example 3

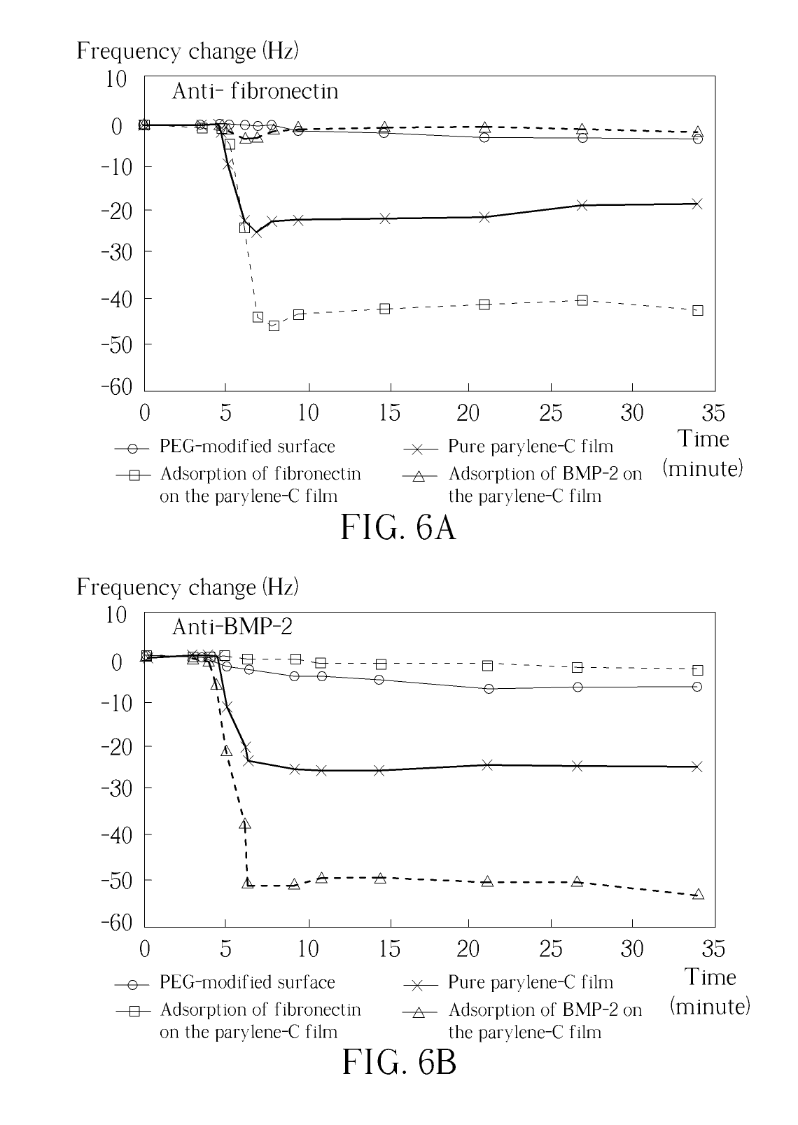

In order to investigate whether the surface substance adsorbed on the parylene-C film retains the biological activity of the original protein, the present example exposes the surface of the adsorbed fibronectin or BMP-2 layer to human BMP-2 antibody and human fibronectin antibody, and then detects the binding affinity of adsorbed BMP-2 or fibronectin to the corresponding antibody. The binding affinity of the adsorbed BMP-2 or fibronectin toward corresponding antibodies is examined by using a QCM. FIG. 6A and FIG. 6B are QCM analyses of the binding affinity of a specific antibody according to the third embodiment of the present invention. As shown in FIG. 6A, a high binding efficiency is exhibited by the human fibronectin antibody (21.0±1.4 ng/cm²) on the parylene-C film adsorbed fibronectin and a low binding efficiency with respect to parylene-C film adsorbed BMP-2. As shown in FIG. 6B, a high binding efficiency is exhibited by the human BMP-2 antibody (35.1±2.3 ng/cm²) on the parylene-C film adsorbed BMP-2 and a low binding efficiency with respect to parylene-C film adsorbed fibronectin. The high binding efficiency observed between the adsorbed proteins and the corresponding human antibodies demonstrates that, although a certain degree of denaturation may occur, the important biological activity of the adsorbed proteins, e.g., specificity toward the corresponding antibody, is maintained during the adsorption process. The parylene-C film and PEG-modified surfaces of unabsorbed fibronectin or BMP-2 exhibit low binding efficiency.

### Example 4

In the present example, a protein solution containing BMP-2 and fibronectin in different proportions (for example, 1:0, 10:1, 1:1: 1:10 and 0:1) is used to investigate combined and competing adsorption of BMP-2 and fibronectin on the same surface of parylene-C film. The binding affinity for a specific antibody is cross-examined by adsorbing varying ratios of BMP-2 and fibronectin on the surfaces of parylene-C film. A combinatorial approach with cross-examination of the adsorbed protein mixture surfaces is subsequently performed by exposing the surfaces to human BMP-2 antibody and human fibronectin antibody. Table 1 shows the results of QCM analysis of the binding affinity of parylene-C film adsorbed BMP-2 and fibronectin in different proportions with a specific antibody. As shown in Table 1, a high binding efficiency of the human BMP-2 antibody on the mixture surfaces is observed with increasing BMP-2 ratio, and the binding efficiency of the human fibronectin antibody similarly increases with increasing fibronectin content. The binding ratios of the two antibodies are calculated and normalized with respect to the combinatorial results, resulting in values of 1, 0.97, 0.51, 0.16 and 0 for BMP-2 and 1, 0.90, 0.57, 0.12 and 0 for fibronectin. These ratios are proportional to the individual solution concentrations of BMP-2 or fibronectin and are well correlated with the BMP-2/fibronectin mixture ratios of 1:0, 10:1, 1:1, 1:10, and 0:1. The results demonstrate that the protein composition on a surface of parylene-C film may be controlled by competing adsorption of multiple proteins. The resulting protein composition may be predicted and controlled by tuning the composition of the protein mixture in the solution phase.

TABLE 1

| Mixing ratio in the solution | Binding ratio of | | Binding constant | Binding ratio of | | Binding constant |
|---|---|---|---|---|---|---|
| (BMP-2:fibronectin) | anti-BMP-2 | | (10⁵ mL/μg) | anti-fibronectin | | (10⁵ mL/μg) |
| 1:0 | 1.00 ± 0.05 | (1.00)^{a} | 8.1 ± 0.3 | 0.00 ± 0.02 | (0.00)^{b} | — |
| 10:1 | 0.97 ± 0.07 | (0.91) | 7.9 ± 0.3 | 0.12 ± 0.05 | (0.09) | 4.5 ± 0.6 |
| 1:1 | 0.51 ± 0.10 | (0.50) | 7.1 ± 0.5 | 0.57 ± 0.09 | (0.50) | 5.2 ± 0.5 |
| 1:10 | 0.16 ± 0.06 | (0.09) | 6.7 ± 0.4 | 0.90 ± 0.04 | (0.91) | 5.1 ± 0.2 |
| 0:1 | 0.00 ± 0.03 | (0.00) | — | 1.00 ± 0.03 | (1.00) | 5.1 ± 0.3 |

^{a}Theoretical value of anti-BMP-2 in the bracket.
^{b}Theoretical value of anti-fibronectin in the bracket.

### Example 5

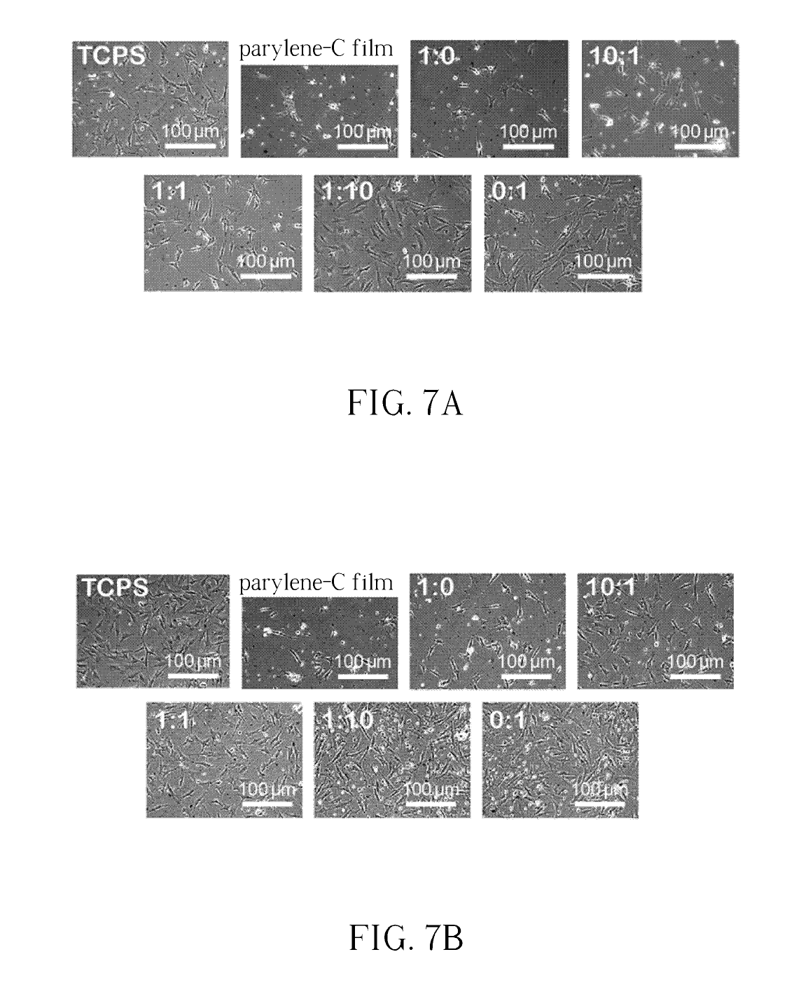

The biological material produced by the present invention may be used for cell culture and can be combined with Example 4 of the present invention, wherein the cell culture may be used in vivo or in vitro. According to an embodiment of the present invention, the surfaces of cell culture plates (12 well) are modified using the aforementioned parylene-C film and protein adsorption procedures of Example 4 of the present invention before cell culture. Next, pADSCs isolated from subcutaneous adipose tissues are seeded at a density of 1×10⁴ cells/cm² and cultured on the modified surfaces of cell culture plates. Cell culture is performed in basal proliferation medium including Dulbecco's modified Eagle's medium with nutrient mixture F-12 containing 10% fetal bovine serum, 100 kU/L penicillin, 100 mg/L streptomycin and 1.5 mg/L amphotericin B at 5% CO₂, 37° C. and 100% humidity. In one embodiment, the resulting samples conducted on the cell culture for 24 hours and 72 hours are observed and photographed using an optical microscope at 100× magnification. For comparison, the present invention uses tissue culture polystyrene (TCPS) and pure parylene-C film for cell culture as control surfaces. FIG. 7A and FIG. 7B are optical microscopic images of the cell culture after 24 hours and 72 hours, respectively, according to the fifth embodiment of the present invention. As shown in FIG. 7A, the cell growth and proliferation of pADSCs on the surfaces of the parylene-C film adsorbed BMP-2 and fibronectin (varying ratios of 1:0, 10:1, 1:1, 1:10, and 0:1) are examined after cell culture for 24 hours, and indicated an increasing trend of cell viability. The number of pADSCs is increased with fibronectin concentration, which is consistent with the biological function of the enhancement of proliferation by fibronectin. As shown in FIG. 7B, continued cell growth and proliferation of pADSCs on the surfaces of the parylene-C film adsorbed BMP-2 and fibronectin varying in different ratios are further verified by examination 72 hours after cell culture. The results provide evidence of the regulation of the biological response by adsorption and the sustained effectiveness of the adsorbed fibronectin protein.

### Example 6

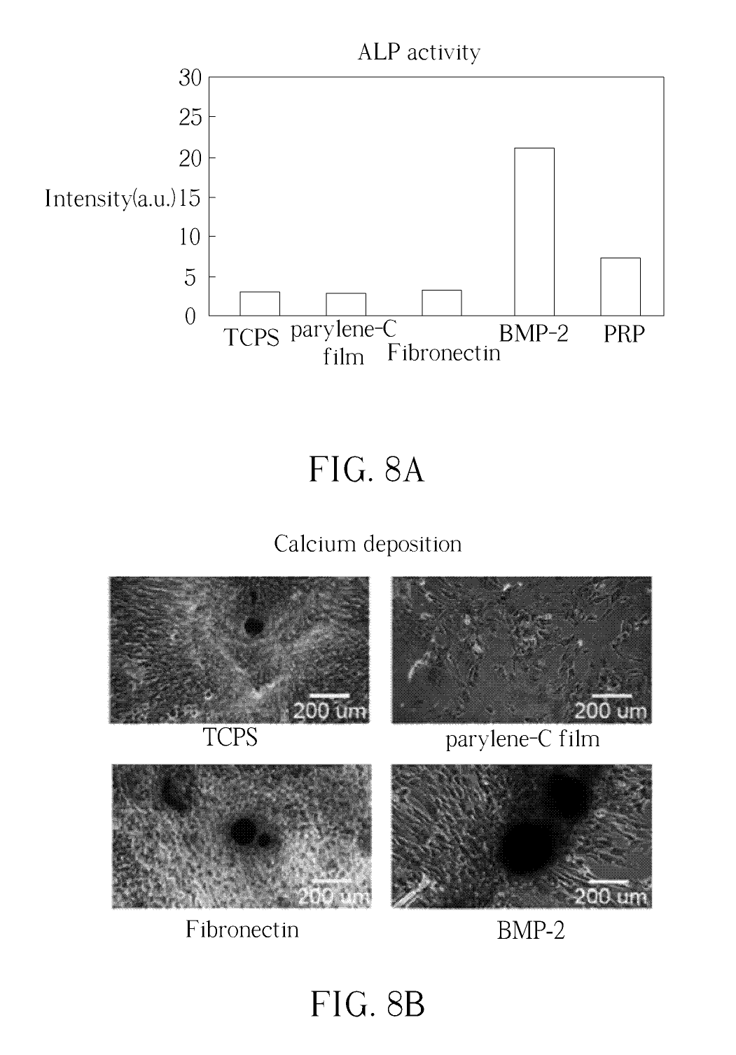

In the present example, pADSC cells are cultured on the biological material of Example 1 to investigate whether adsorbed BMP-2, fibronectin and/or PRP could induce osteogenesis, and may be used in combination with the above-mentioned examples. Osteogenesis is investigated by examining alkaline phosphatase (ALP) expression, calcium mineralization (calcium deposition) and osteogenic marker genes, wherein ALP represents the early marker of osteogenesis, wherein calcium mineralization is the characteristics of mature stage of osteogenesis. FIG. 8A is an ALP expression after 10 days of cell culture according to the sixth embodiment of the present invention. As shown in FIG. 8A, pADSCs cultured on the surface of parylene-C film adsorbed BMP-2 and PRP show a significant increase in ALP expression compared to pADSCs cultured on surfaces of pure TOPS and parylene-C film (control surfaces). FIG. 8B is an optical microscope image of calcium mineralization after 21 days of cell culture according to the sixth embodiment of the present invention. As shown in FIG. 8B, pADSCs cultured on the surface of parylene-C film adsorbed BMP-2 show a significant calcium deposition compared to pADSCs cultured on surfaces of pure TOPS and parylene-C film (control surfaces). The above experimental results demonstrate that the present invention indeed induces osteogenesis of cells by adsorbing regulatory factors of osteogenesis (BMP-2 and PRP) on parylene-C film, which is consistent with the biological function of the inducement of osteogenesis by BMP-2 and PRP.

The present invention provides a novel method for manufacturing biological material. The method exploits the simple and intuitive adsorption process to immobilize different functional materials, including bone morphogenic protein-2 (BMP-2), fibronectin, and platelet-rich plasma (PRP), on the parylene-C film. It should be noted that the proposed method of the present invention is mediated by hydrophobic interactions without the use of potentially harmful substances during the modification process, which thereby increases the potential applications of the parylene-C film. Moreover, the biological functions of the functional proteins are effectively mounted on the surface of the parylene-C film.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A biological material, comprising:
a parylene-C film; and
first proteins which are adsorbed on a surface of the parylene-C film.

2. The biological material according to claim 1, wherein the biological material further comprises second proteins different from the first proteins adsorbed on the surface of the parylene-C film.

3. The biological material according to claim 1, wherein the first proteins or the second proteins comprise BMP-2, fibronectin or PRP.

4. A method of manufacturing a biological material, comprising:
providing a substrate;
performing a vapor deposition process such that a parylene-C film is deposited on the substrate; and
providing a protein solution, wherein the parylene-C film is immersed in the protein solution to form a surface substance on the parylene-C film.

5. The method of manufacturing a biological material according to claim 4, further comprising:
after forming the surface substance on the parylene-C film, performing a rinse process to rinse the surface substance.

6. The method of manufacturing a biological material according to claim 4, wherein the surface substance has biological functions.

7. The method of manufacturing a biological material according to claim 6, wherein the biological functions comprise cell proliferation and/or osteogenesis.

8. The method of manufacturing a biological material according to claim 4, wherein the protein solution comprises BMP-2, fibronectin and/or PRP.

9. The method of manufacturing a biological material according to claim 8, wherein the protein solution comprises two or more proteins and has a predetermined ratio.

10. The method of manufacturing a biological material according to claim 9, wherein a composition ratio of the surface substance is the same as the predetermined ratio of the protein solution.

11. The method of manufacturing a biological material according to claim 5, wherein the rinse process comprises:
rinsing the surface substance three times with a phosphate-buffered saline containing Tween-20;
rinsing the surface substance once with a phosphate-buffered saline without Tween-20; and
rinsing the surface substance once with deionized water.

12. The method of manufacturing a biological material according to claim 5, wherein the surface substance is adsorbed on the parylene-C film and the adsorption of the surface substance is irreversible.

13. A use of a biological material according to claim 1, wherein the biological material is used for cell culture.
